(19) United States
(12) Patent Application Publication
(10) Pub. No.: US 2023/0277651 A1
(43) Pub. Date: Sep. 7, 2023

(54) IMMUNOGLOBULIN MEDIATED VACCINATIONS AGAINST VIRAL DISEASES

(71) Applicant: Martin H. Bluth, Southfield, MI (US)

(72) Inventor: Martin H. Bluth, Southfield, MI (US)

(21) Appl. No.: 18/018,201

(22) PCT Filed: Jul. 26, 2021
(86) PCT No.: PCT/US2021/043243

§ 371 Date: Jan. 26, 2023

Related U.S. Application Data
(60) Provisional application No. 63/057,061, filed on Jul. 27, 2020.

(51) Int. Cl. A61K 39/215

(52) U.S. Cl. CPC A61K 39/215; A61K 2039/505

(57) ABSTRACT

Techniques for inhibiting viral disease are provided. The techniques include obtaining a blood product from a convalescent patient, and administering the blood product to a patient at risk of contracting the same type of virus in order to propagate immunoglobulin-based vaccination.

### CROSS-REFERENCE TO RELATED APPLICATIONS

This application claims the benefit of International Application No. PCT/US2021/043243, filed Jul. 26, 2021, which claims priority to the U.S. Provisional Application No. 63/057,061, filed Jul. 27, 2020, the disclosures of which are incorporated herein by reference in their entireties.

### FIELD OF THE INVENTION

The present invention relates generally to immunology and, more particularly relates to vaccination immunology.

### BACKGROUND OF THE INVENTION

Viral infections foster devastating disease that are challenging to treat and, in certain cases, offers little chance of avoiding morbidity and/or mortality. By way of example, Severe Acute Respiratory Syndrome Coronavirus-2 (i.e., SARS-CoV-2 also known as Coronavirus Disease-19 (i.e., COVID-19)), as a representation of viral infection, has affected the globe and was responsible for over 15 million infections resulting in over 600,000 deaths worldwide (as of Jul. 22, 2020). Despite existing potential therapeutic approaches such as, for example, hydroxychloroquine, remdesivir, dexamethasone and antibiotics such as azithromycin, COVID-19 remains a significant morbidity and mortality concern around the world.

Early diagnosis of viral infection can be challenging based on emerging diagnostic approaches that differ from each other (i.e. molecular versus serological testing) and vary due to methodological approaches that can miss certain patients with viral infection (false negative) or falsely diagnose those without infection (false positive).

Existing diagnostic approaches demonstrate that the presence and concentration of antiviral immunoglobulin levels can be associated with potential immunity to subsequent viral infection. By way of example, in SARS-CoV-2/COVID-19 infection, serum or plasma obtained from patients with SARS-CoV-2/COVID-19 infection that have been assayed are shown to have the presence and/or increased levels of total immunoglobulin and/or antigen specific (i.e. SARS-CoV-2/COVID-19) immunoglobulin including immunoglobulin G (IgG) and immunoglobulin M (IgM), immunoglobulin A (IgA) and their respective subclasses (i.e. IgG1, IgG2, IgG3, IgG4), when compared with healthy controls. Although not well described, it is likely that other immunoglobulin isotypes including immunoglobulin E (IgE) and immunoglobulin D (IgD) are also propagated in the immune response to SARS2 as they have been described in other viral infections

Approaches for therapeutic strategies have also been pursued. For example, infusion of convalescent plasma (which is plasma obtained from patients who have been infected with SARS-CoV-2, recovered from their illness and have been shown to propagate anti-SARS immunoglobulin responses (i.e. IgG and/or IgM anti-SARS antibodies) has been instituted as treatment for new patients with mild to severe symptomatic SARS infection. This convalescent plasma serves to potentially “neutralize” circulating virus from binding to specific receptors and avoid infecting healthy cells. Also, vaccine therapy (which elicits the patient's immune response against any of a number of putative SARS-CoV-2 viral antigens including, for example, the structural proteins comprising spike (S), envelope (E), membrane (M), and nucleocapsid (N), and fragments thereof, expressed by SARS-CoV-2/COVID that may act as antigens to activate neutralizing antibodies and generate defensive response) has been described.

However, conventional approaches to long lasting treatment of viral infections such as SARS/COVID have, thus far, been largely unsuccessful.

### SUMMARY OF THE INVENTION

In one aspect, the present invention provides a method of inhibiting a viral infection. The method comprises a) obtaining a blood product from a first subject wherein the blood product has anti-viral immunoglobulins against a virus, and b) administering an immunization effective amount of the blood product obtained from the first subject to a second subject who is at risk of becoming infected with the same or similar type of virus. The viral infection in the second subject is inhibited. In one embodiment, the blood product is selected from the group consisting of: serum; plasma; and one or more immunoglobulins with or without their corresponding soluble receptors, and fragments thereof. In one embodiment, the first subject has recovered from an infection by the virus. In another embodiment, the first subject has been previously immunized against the virus. Inhibition of a viral infection includes preventing a viral infection. In some embodiments, the blood product is irradiated prior to administration to the second subject.

In some embodiments, the virus is a coronavirus, HIV, H1N1, H5N1, Powassan virus, Zika virus, chikungunya virus, dengue virus, West Nile virus, herpesvirus, norovirus, parvovirus, human papillomavirus, respiratory syncytial virus, influenza virus, SARS-CoV-2, MERS-CoV, avian flu virus, Ebola virus, influenza A virus, SARS virus, hepatitis virus, measles virus, rubella virus, chickenpox virus, or yellow fever virus.

In some embodiments, the methods further comprise removing a corresponding amount of serum and/or plasma from the second subject prior to administering the serum and/or plasma obtained from the first subject. In some embodiments, the methods further comprise removing a corresponding amount of serum and/or plasma from the second subject substantially simultaneously with administering the serum and/or plasma obtained from the first subject.

In some embodiments, the one or more immunoglobulins comprise at least one of immunoglobulin G (IgG), immunoglobulin M (IgM), immunoglobulin A (IgA), immunoglobulin E (IgE), and immunoglobulin D (IgD) and fragments thereof. In some embodiments, the fragments are selected from the group consisting of antibody binding fragment Fab, antibody subclasses IgG1, IgG2, IgG3, IgG4, and combinations thereof.

In some embodiments, an immunization effective amount of one or more immunoglobulins and one or more soluble receptors corresponding to each immunoglobulin comprises an immunization effective amount of anti-virus specific immunoglobulin. An immunization effective amount is one that inhibits contracting a virus and/or attenuates the symptoms of a virus.

In one embodiment, the virus is SARS-CoV-2/CoViD-19, the blood product is serum/plasma and the immunization effective amount is about 5 ml to about 200 mL. In one embodiment, the virus is SARS-CoV-2/CoViD-19, the blood product is serum/plasma and the immunization effective amount is about one picogram per kilogram per day to about four grams per kg per day of antigen-specific immunoglobulin. In one embodiment, the virus is SARS-CoV-2/CoViD-19, the blood product is serum/plasma and the immunization effective amount is administered at a single or multiple interval sequence from single or multiple subjects who have recovered from a type of virus infection.

In some embodiments, a blood product obtained from the first subject is administered to a plurality of subjects at risk of contracting the same or similar type of virus. In some embodiments, the methods further comprise administering conventional vaccination protocols including immunological stimulators and/or boosters of specific immune pathways.

In some embodiments, the methods further comprise administering a chimeric or engineered form of the antibody. In some embodiments, the engineered form of the antibody is an Fc component fused with a SARS-Cov-2/CoViD-19 viral antigen in place of Fab.

In one aspect of the invention, methods of treating viral infections are provided. The methods comprise the steps of: obtaining an amount of at least one blood product from at least a first patient with a type of virus infection; and administering the at least one of blood product obtained from the at least first patient to at least a second patient with the same type of virus, wherein administering the at least one blood product obtained from the at least first patient to at least a second patient with the same type of virus comprises administering a therapeutically effective amount of one or more immunoglobulins and/or one or more soluble receptors corresponding to each immunoglobulin, and wherein administering a therapeutically effective amount of one or more immunoglobulins and/or one or more soluble receptors corresponding to each immunoglobulin comprises enabling reconstitution of a humoral immune system of the at least a second patient to treat the virus. In some embodiments, the immunoglobulins with or without their corresponding soluble receptors can alternatively be purified from serum/plasma as a means to administer the components without the additional volume inherent in total serum/plasma. In one embodiment, the type of virus comprises SARS2/COVID-19. In some embodiments, the method further comprises removing a corresponding amount of at least one of serum and plasma from the second patient with the same type of virus. In some embodiments, removing the corresponding amount of at least one of serum and plasma from the second patient with the same type of virus is performed prior to administering the at least one of serum and plasma obtained from the first patient to the second patient with the same type of virus, or removing the corresponding amount of at least one of serum and plasma from the second patient with the same type of virus is performed substantially simultaneously to administering the at least one of serum and plasma obtained from the first patient to the second patient with the same type of virus. In some embodiments, the plasma and serum are interchangeable with one another with respect to treatment of the virus. In some embodiments, the one or more immunoglobulins comprise at least one of immunoglobulin G (IgG), immunoglobulin M (IgM), immunoglobulin A (IgA), immunoglobulin E (IgE), and immunoglobulin D (IgD). In some embodiments, the one or more immunoglobulins comprise at least one of immunoglobulin specific for one or more viral antigens. In some embodiments, a therapeutically effective amount of one or more immunoglobulins and one or more soluble receptors corresponding to each immunoglobulin comprises a therapeutically effective amount of anti-virus specific immunoglobulin. In some embodiments, enabling reconstitution of a humoral immune system of the at least a second patient comprises neutralizing and/or destroying free virus, and/or virus infected cells, or comprises alleviating virus progression. In some embodiments, administering the at least one of convalescent serum and plasma obtained from the first patient to the second patient further comprises repeatedly administering the at least one of serum and plasma obtained from the first patient to a plurality of patients with the same type of virus. In some embodiments, the at least one of convalescent serum and plasma obtained from the first patient is repeatedly administered to the plurality of patients with the same type of virus substantially simultaneously. In some embodiments, obtaining the amount of at least one of serum and plasma comprises obtaining respective amounts of at least one of serum and plasma from a plurality of patients with the same type of virus. In some embodiments, the respective amounts of at least one of serum and plasma are obtained from the plurality of patients with the same type of virus substantially simultaneously and administered to one or more patients recipients in need thereof. In some embodiments, the at least one of serum and plasma obtained from the first patient is administered as at least one of a single dose and repeated doses. In some embodiments, the immunoglobulins with or without their corresponding soluble receptors can alternatively be purified from serum/plasma as a means to administer the components without the additional volume inherent in total serum/plasma. And, serum/plasma obtained from any patient could be irradiated prior to administration to a blood group compatible recipient. In some embodiments, the methods can be augmented by conventional vaccination protocols in addition to immunological stimulators and/or boosters of specific immune pathways (e.g. cytokine [IL-10] based) to maximize the efficiency of the immunoglobulin mediated anti-viral responses described herein and anti-viral responses in general.

These and other features, objects and advantages of the present invention will become apparent from the following detailed description of illustrative embodiments thereof, which is to be read in connection with the accompanying drawings.

### BRIEF DESCRIPTION OF THE DRAWINGS

FIG. 1 is a diagram illustrating a typical immunoglobulin (e.g. IgG, IgM, IgA, IgE, IgD) structure, according to an embodiment of the present invention.

FIG. 2 is a diagram illustrating anti-idiotypic antibody generation in at-risk patients exposed to convalescent plasma, where the anti-idiotypic antibody is similar to the antigen, according to an embodiment of the present invention.

FIG. 3 is a diagram illustrating anti-anti-idiotypic antibody generation in at-risk patients exposed to convalescent plasma, where the anti-anti-idiotypic antibody represents vaccination to the virus, as evidenced by recognizing the native virus, yet not generated by the native virus by conventional vaccination approaches, according to an embodiment of the present invention.

FIG. 4 is a diagram of a chimeric or engineered form of the antibody (i.e., Fc component fused with a SARS/CoViD-19 viral antigen in place of Fab).

FIG. 5 is a diagram of an Antibody IgG structure and cleavage sites for fragmentation. Examples of useful antibody fragments include half-IgG, Fab, F(ab′)2 and Fc; they can be produced by reduction of hinge region disulfides or digestion with papain, pepsin or ficin proteolytic enzymes.

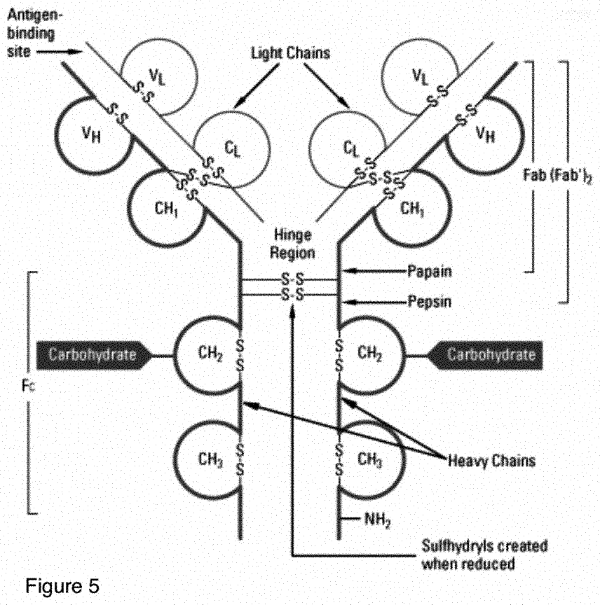

### DETAILED DESCRIPTION OF THE INVENTION

In one aspect, the present invention provides methods for immunization against viral infections/diseases in subjects (i.e., patients) in need thereof. In another aspect, the present invention provides treatment of subjects (i.e., patients) with a viral infection.

The term “patient” as used herein refers broadly to mammalian subjects, preferably humans, receiving medical attention (e.g., diagnosis, monitoring, etc.).

Examples of some viral diseases for which the methods of the invention are suited include diseases caused by Coronaviruses (e.g., Coronavirus-2), HIV, H1N1, H5N1, Powassan virus, Zika virus, chikungunya virus, dengue virus, West Nile virus, herpesviruses, norovirus, parvovirus, human papillomavirus, respiratory syncytial virus, influenza virus, and agents associated with neurodegenerative diseases and Transmissible Spongiform Encephalopathies (e.g., Creutzfeld-Jacob Disease, kuru and bovine spongiform encephalopathy). Examples of some viral diseases for which the methods of the invention are suited include Swine Influenza and Avian Influenza, Ebola virus disease, influenza A, SARS, hepatitis, measles, rubella, chickenpox, and yellow fever. Examples of illnesses caused by Coronaviruses include Middle East Respiratory Syndrome (MERS-CoV), Severe Acute Respiratory Syndrome (SARS-CoV) and Coronavirus Disease 2019 (COVID-19), coagulopathy, kidney injury, cardiomyopathy and neurological impairments.

Swine influenza is a respiratory disease that occurs in pigs that is caused by the Influenza A virus. Influenza viruses that are normally found in swine are known as swine influenza viruses (SIVs). The known SIV strains include influenza C and the subtypes of influenza A known as H1N1, H1N2, H3N1, H3N2 and H2N3. Avian Influenza, H5N1, is the highly pathogenic influenza A virus subtype.

### Inhibiting Viral Infection

In one aspect of the present invention, a viral infection is inhibited in a patient in need thereof. A patient in need thereof is a subject that is at-risk of contracting a viral infection. In particular, a pharmaceutical composition of the present invention is administered prophylactically to an individual who is at-risk of contracting a viral infection/disease. Examples of individuals who are at-risk may have one or more of the following characteristics: are elderly; are under stress and/or are depressed; have weakened immune systems or are immunocompromised; and/or have been in contact with a person having, or suspected of having, the viral disease.

In this aspect, the present invention, in illustrative embodiments thereof, provides techniques for the immunization against viral infections. In one embodiment, the methods comprise the administration of a pharmaceutical composition to a patient in need thereof in an amount which is effective for the immunization against a viral infection, thereby inhibiting a viral infection. Inhibiting a viral infection includes preventing a viral infection and/or attenuating the symptoms of a viral infection. The method comprises obtaining a blood product from a first subject. The first subject is known to have been infected with a certain type of virus and has recovered from such infection; or the first subject has been immunized against such virus. The first subject is also known as the “convalescent patient” or patient A. That is, this convalescent patient has demonstrated the presence of anti-viral immunoglobulins (e.g. IgG/IgM) in his/her serum/plasma either i) due to a viral infection that has cleared and is no longer infectious to others, or ii) due to previous immunization. The convalescent patient has humoral immunity against the viral pathogen at issue, and is a source of specific antibodies. An immunization effective amount of the blood product (obtained from the convalescent patient) is administered to a second patient who is at-risk of becoming infected with the same or similar (e.g., same family, genus, species) type of virus (i.e., at-risk patient (patient B)). The blood product of the convalescent patient has not been shown infect the second patient. The second patient is blood group compatible with the convalescent patient. In some embodiments, the blood product is irradiated prior to administration. The anti-viral immunoglobulins from the first patient will serve as a vaccine to stimulate the immune response of the second patient to propagate new antiviral immunoglobulins that will protect the second patient from being infected by the same or similar virus.

Examples of suitable blood products include serum; plasma; and one or more immunoglobulins (with or without their corresponding soluble receptors), and fragments thereof. Some examples of immunoglobulins include immunoglobulin G (IgG), immunoglobulin M (IgM), immunoglobulin A (IgA), immunoglobulin E (IgE), and immunoglobulin D (IgD). Several types of antigen-binding fragments of immunoglobulins are suitable; each fragment contains at least the variable regions of both heavy and light immunoglobulin chains (VH and VL, respectively) held together (typically by disulfide bonds) so as to preserve the antibody-binding site. Examples of immunoglobulin fragments suitable for the invention include antibody binding fragments such as Fab, Fab(2), either individually or in combination thereof, and subclasses [IgG1, IgG2, IgG3, IgG4], either alone or in combination. Examples of antigen specific antigens include IgG3 and/or IgE, either alone or in combination; IgG; IgM; IgA; IgE; and/or IgD; and subclasses (i.e. IgG1, IgG2, IgG3, and/or IgG4).

That is, immunoglobulins specific for particular viral antigens which are missing in the at-risk patient are supplied by the convalescent blood product (e.g., serum or plasma) of a patient who has recovered from the same type of virus as a means to propagate new antiviral immunoglobulins in the at-risk subject.

The blood product(s) (e.g., serum/plasma) obtained from a convalescent patient functions as a type of hyperimmune anti-virus blood product to stimulate the immune system of an at-risk patient who has not yet encountered the wild type virus. Consequently, maturation of both anti-idiotypic and anti-anti-idiotypic antibodies are promoted in the at-risk patient. Hyperimmune refers to a physiologic state where there is a high concentration of antibodies produced in reaction to repeated injections of an antigen. These methods effectively immunize the at-risk patient against the same or similar virus and/or conformational shape.

An “immunization effective amount” of blood product(s) in the methods of the present invention is defined herein as an amount sufficient to produce a measurable inhibition and/or attenuation of a viral disease and/or a measurable diagnostic effect of anti-virus antibodies in the patient receiving the convalescent blood product. A “diagnostic effect” herein means that the patient receiving the blood product(s) demonstrates that they made their own antiviral antibodies after receiving the blood product(s).

The anti-viral effect of a particular blood product depends upon the presence and concentration of antigen-specific immunoglobulins, rather than total immunoglobulin levels. The content and characteristics of immunoglobulin levels can vary from convalescent patient to convalescent patient. That is, purified antivirus immunoglobulins from a particular convalescent patient may contain high levels of antivirus immunoglobulins to mediate an effective vaccination response; whereas purified anti-virus immunoglobulin from a different convalescent patient may not. Thus, in some embodiments of the present invention, convalescent blood products (e.g., serum or plasma) are modified, manipulated, purified and/or concentrated to increase the anti-virus antibodies present in the convalescent blood product so to increase the efficacy needed to propagate an “immunoglobulin” based vaccination (as opposed to typical “virus” based vaccination) via the anti-idiotypic network.

Thus, in the embodiment where the blood product administered is convalescent plasma/serum, the effective amount of convalescent plasma/serum administered to an at-risk patient will depend upon the level of antigen-specific immunoglobulins in such plasma/serum. A sample of the convalescent plasma/serum can be tested to ascertain the level of antigen specific immunoglobulin and be administered accordingly.

By way of example, for immunization, the amount of antigen-specific immunoglobulin is administered to an at-risk patient in a dosage amount of between about one picogram (pg) per kilogram (kg) per day (d) (pg/kg/d) to about three grams (g) per kg per d (g/kg/d), wherein the kg value represents the weight of the patient. Examples of other lower boundaries of this range include about 1 ng/kg/d, about 1 μg/kg/day, about 1 mg/kg/day and about 1 g/kg/day. Examples of other upper boundaries of this range include about 1 g/kg/day, 1.5 g/kg/day, about 2 g/kg/day and about 2.5 g/kg/day. Each lower boundary can be combined with each upper boundary to define a range. The lower and upper boundaries should each be taken as a separate element. Such dosage amount is the antigen-specific immunoglobulin itself, which can be administered on its own or in the presence of plasma/serum. In one embodiment, a typical example of a dosage of antigen-specific immunoglobulin is about 200 mg/kg/day to about 600 mg/kg/day, more typically about 300 mg/kg/day to about 500 mg/kg/day, even more typically, about 400 mg/kg/day. In one embodiment, typically, the antigen-specific immunoglobulin is administered for about 2 to 7 days, more typically, from about 3 to 6 days, even more typically for about 5 days. In another embodiment, a typical example of a dosage amount of antigen-specific immunoglobulin is between about 75 micrograms (μg) per kg per d (m/kg/d) to about 4000 milligrams (mg) per kg per d (mg/kg/d).

In one embodiment, for example, if the virus is SARS-CoV-2/CoViD-19, and the blood product is plasma, an immunization effective amount of such plasma is in the range of about 5 mL to about 450 mL. Examples of other lower boundaries of this range include about 10 mL, about 20 mL, about 30 mL, about 40 mL, about 50 mL, about 60 mL, about 70 mL, about 80 mL, about 90 mL and about 100 mL. Examples of other upper boundaries of this range include about 100 mL, 110 mL, about 120 mL, about 130 mL, about 140 mL, about 150 mL, about 160 mL, about 170 mL, about 180 mL, about 190 mL and about 200 mL. Each lower boundary can be combined with each upper boundary to define a range. The lower and upper boundaries should each be taken as a separate element.

In embodiments where plasma and/or serum is administered, plasma can be removed from patient B receiving serum/plasma from patient A if volume overload is a concern for patient B (e.g., typically, an equal amount of plasma is removed as being administered). In some embodiments, a corresponding amount of serum and/or plasma is removed from the second subject prior to administering the serum and/or plasma obtained from the first subject. In some embodiments, a corresponding amount of serum and/or plasma is removed from the second subject substantially simultaneously with administering the serum and/or plasma obtained from the first subject. In some embodiments, instead of administering total serum/plasma to the patient at-risk, antigen-specific immunoglobulins are purified from the serum/plasma of patient A and administered to patient B. In such manner, the additional volume inherent in total serum/plasma is avoided. The immunoglobulins can be administered with or without their corresponding soluble receptors.

In some embodiments, a blood product obtained from the first subject is administered to a plurality of subjects at-risk of contracting the same or similar type of virus. In some embodiments, the blood product is administered at a single or multiple interval sequence from single or multiple convalescent patients. For example, this approach can be repeated and administered in tandem from other patients who have recovered from the same type of viral infection. For example, convalescent blood product(s) (e.g., serum or plasma) containing anti-viral antibodies can be obtained from a third or fourth patient with anti-virus antibodies (patient D or E) and administered to the at-risk patient (patient B) in no particular set sequence. Moreover, such administration can be administered as a single dose or as repeated doses.

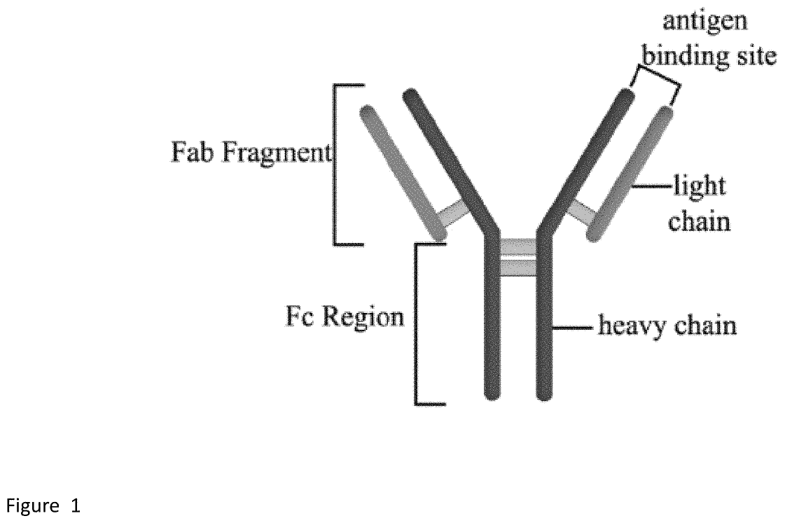

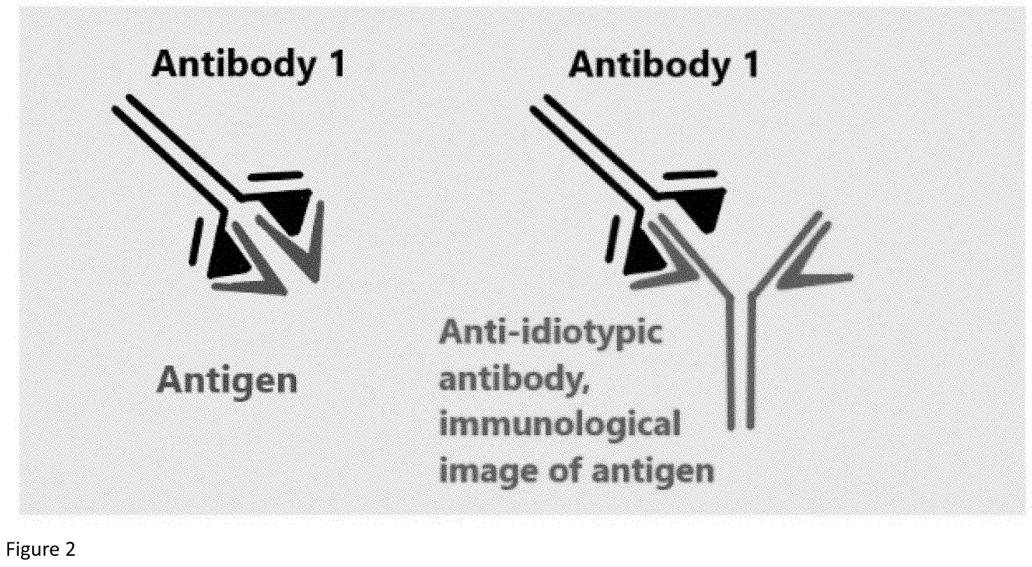

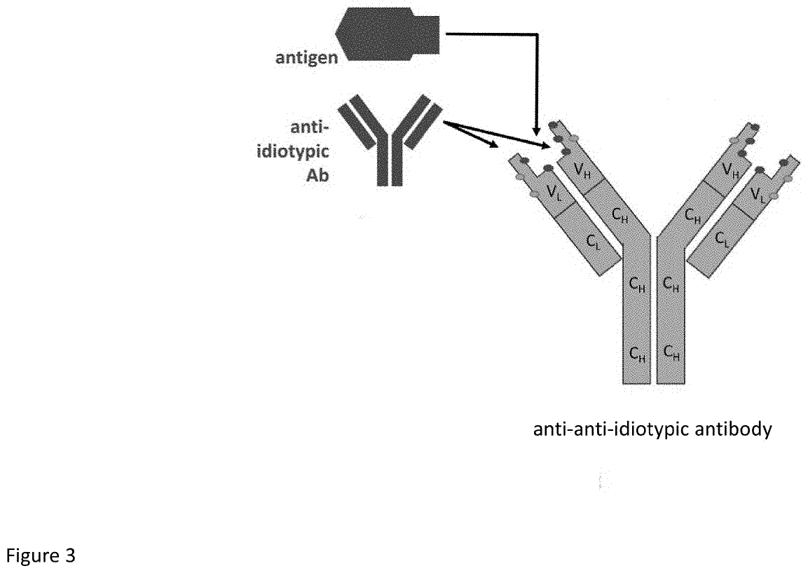

Without wanting to be bound by a mechanism of action, it is believed that the anti-viral immunoglobulins present in the convalescent blood product from recovered patients (e.g., patient A) will stimulate the maturation of anti-idiotypic antibodies (which look like the viral antigens) in the at-risk patient (i.e., patient B), with the subsequent generation of anti-anti-idiotypic antibodies in patient B (which are similar to the antibody response propagated after conventional vaccination approaches) to effectively neutralize native virus that may infect patient B at a later time. As such, one or more embodiments of the invention will facilitate and enable the reconstitution of patient B's humoral immune system to destroy native virus and obviate any infection. In other words, it is believed that anti-idiotype antibodies serve as a vaccine which mirrors the three-dimensional shape of the virus itself. For example, the antibody binding fragment (Fab) of the anti-viral immunoglobulin (FIG. 1) obtained from a blood product of the first patient, once administered (e.g., injected via blood stream, intramuscularly, topically administered through skin or aerosolization), to the second patient is seen as “foreign” by second patient. The three-dimensional structure of the Fab of the first patient is referred to as the idiotype. The immune system of the second patient now mounts his/her own antibodies to recognize the Fab region of the first patient, referred to as anti-idiotypic antibodies (FIG. 2). These newly formed anti-idiotype antibodies have the same three-dimensional shape as the virus itself. Subsequently, these anti-idiotypic antibodies, which are replicas of the virus itself, now foster new antibodies to recognize these anti-idiotypic Fab regions to make new antibodies, referred to as anti-anti-idiotypic antibodies (FIG. 3). These anti-anti-idiotypic antibodies are effectively anti-viral immunoglobulin responses and mirror standard viral immunization approaches.

Before the present invention, the roles of antiviral immunoglobulins (e.g., IgG, IgM, IgA, IgE, IgD) and, their respective receptors, have not been investigated as a vaccination intervention for viral disease. In the methods of the present invention, anti-viral antibodies obtained from convalescent blood products (e.g., serum or plasma) have a protective effect in viral disease and serve as a potent vaccination mechanism. Without wanting to be bound to a mechanism of action, it is believed that antiviral immunoglobulins specific to virus antigens kill virus infected cells by antibody-dependent cell-mediated cytotoxicity (ADCC) as well as mediate killing virus infected cells by Complement (C)-mediated cytotoxicity, as well as mediate killing of virus/virus infected cell by nitric oxide, as well as inhibit virus infection by neutralizing the virus from infecting healthy cells. For example, IgG, IgM, IgA, IgE, IgD and/or their respective receptors provide an immunization effect, by the propagation of anti-idiotypic networks generating effective vaccination against virus, in virus infection, without ever introducing the virus into the patient.

Anti-idiotypic and anti-anti-idiotypic antibody responses have previously been described in other viral diseases (e.g. HIV-1). Regarding HIV-1 disease, Keay et al. (1992) detected anti-CD4 anti-idiotype antibodies, which are antibodies which antigenically mimic HIV-1 epitopes, in sera of volunteers immunized with recombinant gp160, suggesting that molecular mimicry may enhance an immune response to the original antigen. Others (Deckert et al., 1996) have found that vaccination with monoclonal anti-CD4 antibody, which mimics an epitope of gp120, was able to induce an immune response that inhibits gp120 binding to CD4. Furthermore, Kang et al. (1992) demonstrated that primates immunized with an (anti-HIV-1) anti-idiotype monoclonal antibody were able to neutralize HIV-1. However, in viral disease, the roles of antiviral immunoglobulins (IgG, IgM, IgA, IgE, IgD) and, where applicable, their respective receptors in viral disease have not, thus far, been investigated as a vaccination intervention for viral disease. In the present invention, it has surprisingly been found that immunoglobulins such as, for example, IgG, IgM, IgA, IgE, IgD and/or their respective receptors, can generate effective vaccination against a virus by the propagation of anti-idiotypic networks, without ever introducing the virus into the patient.

In some embodiments, the methods of the present invention is augmented by conventional vaccination protocols including, for example, immunological stimulators and/or boosters of specific immune pathways (e.g. cytokine [IL-10] based or Th1 based adjunctive or augmentative vaccination protocols) to maximize the efficiency of the immunoglobulin mediated anti-viral responses described herein and anti-viral responses in general. Such administration can be administered as a single dose or as repeated doses.

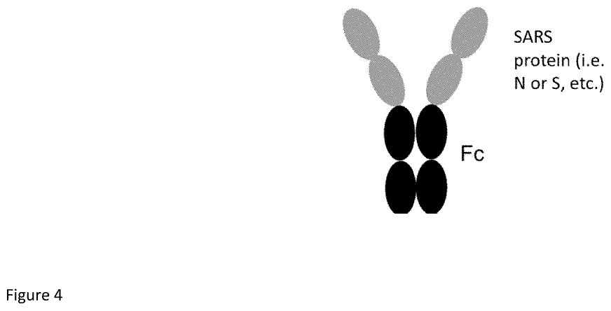

In some embodiments, a chimeric or engineered form of the antibody is administered. For example, see FIG. 4 where an Fc component is fused with a SARS/CoViD-19 viral antigen instead of a Fab. (See also Deckart (1996) Fc-HIV in place of Fab region as immunogen for HIV-1.)

### Treating Viral Infection

In one aspect of the present invention, a viral infection is treated in a patient in need thereof. A patient in need thereof is a subject that currently has a viral infection. In particular, a pharmaceutical composition of the present invention is administered in a therapeutically effective amount. A therapeutically effective amount is defined as an amount that is able to inhibit/attenuate the symptoms of the viral infection.

By way of example, for treatment of a viral infection/disease, the amount of antigen-specific immunoglobulin is administered to an infected patient in a dosage amount of between about one picogram (pg) per kilogram (kg) per day (d) (pg/kg/d) to about three grams (g) per kg per d (g/kg/d), wherein the kg value represents the weight of the patient.

A benefit of the methods of the present invention is that it can be utilized immediately, from existing stored (frozen) stockpiles of convalescent plasma from patients who are now healthy (e.g., recovered SARS/COVID patients), to begin treating and/or immunizing other patients and is not subject to the delay in conventional vaccine development. The convalescent serum/plasma would be administered according to (ABO) blood type compatibility and, in some embodiments, can be irradiated to effectively destroy any possibility of residual viral particles from causing infection in a recipient.

### Techniques Used in the Methods

The use of biotechnology to generate proteolytically and/or recombinant-derived Fab/Fab(2) containing anti-antigen antibody fragments or total Ig anti-antigen antibody has important beneficial properties. First, the use of biotechnology subverts the possibility of transmitting blood-born diseases or pathogens which may be a component of a non-fractionated plasma unit. Biotechnology allows researchers and clinicians to isolate the desired Fab/Fab(2) containing anti-antigen antibody fragments or total Ig anti-antigen antibody. The use of pooled serum is thus avoided.

Second, the use of biotechnology allows researchers to make selective preparations that include only the classes of immunoglobulin molecules, or parts thereof, that are needed for immunization or treatment. The ability to selectively include only certain classes of immunoglobulin molecules is beneficial when the patient receiving the immunization or treatment has a negative reaction to certain classes of immunoglobulin molecules, but not others. For example, a patient may have an adverse allergic reaction to immunization or treatment with IgA. Thus, according to the teachings of the present invention, the patient can receive immunization or treatment with a preparation in which IgA is selectively absent. This selectivity cannot be obtained with isolated fractions because pooled donor serum will likely contain a plurality of classes of immunoglobulin molecules, in varying amounts.

Third, the use of biotechnology allows researchers to produce a quantifiable preparation of Fab/Fab(2) containing anti-antigen antibody fragments or total Ig anti-antigen antibody. As described above, researchers can prepare selective batches of Fab/Fab(2) containing anti-antigen antibody fragments or total Ig anti-antigen antibody fragments. As such, researchers may also control the amount of Fab/Fab(2) containing anti-antigen antibody fragments or total Ig anti-antigen antibody in each batch. The ability to control batch amounts allows for careful monitoring and control of immunization or treatment. In contrast, immunization or treatment with isolated donor serum does not allow for such control. Donor serum will likely contain a predictable amount of each class of immunoglobulin molecule, however, it is not practically possible to quantify that amount for each immunization or treatment. Thus, physicians administering the immunization or treatment may only have an estimate of the quantity of each immunoglobulin heavy chain isotype (i.e., IgG, IgM, IgA, IgE, IgD) and class (i.e., IgG1, IgG2, IgG3, IgG4).

The Fab/Fab(2) containing anti-antigen antibody fragments or total Ig anti-antigen antibody obtained may then be prepared as part of a solution, the solution to be administered to a patient as described below. The solution of Fab/Fab(2) containing anti-antigen antibody fragments or total Ig anti-antigen antibody may comprise less than or equal to about 95 weight percent (wt. %) Fab/Fab(2) containing anti-antigen antibody fragments or total Ig anti-antigen antibody, based on the total weight of the solution. Further, the solution of Fab/Fab(2) containing anti-antigen antibody fragments or total Ig anti-antigen antibody may comprise between about 1 to about 50 wt. % Fab/Fab(2) containing anti-antigen antibody fragments or total Ig anti-antigen antibody fragments, based on the total weight of the solution.

Fab/Fab(2) containing anti-antigen antibody fragments or total Ig anti-antigen antibody is then provided for immunization or treatment of a patient. For example, the manufacturer might provide the obtained Fab/Fab(2) containing anti-antigen antibody fragments or total Ig anti-antigen antibody to a clinician for administering to a patient. While the present method described herein is presented in discrete steps and the description highlights different entities performing different steps, it is to be understood that according to the teachings herein, each of the steps may be performed independently, or concurrently, and in any combination. Further, it is to be understood that any of the steps, independently or in combination, may be performed by a single entity or any combination of entities. By way of example only, in an alternative embodiment, a clinician prepares Fab/Fab(2) containing anti-antigen antibody fragments or total Ig anti-antigen antibody and provides the Fab/Fab(2) containing anti-antigen antibody fragments or total Ig anti-antigen antibody for immunization or treatment of a patient. The patient may obtain the Fab/Fab(2) containing anti-antigen antibody fragments or total Ig anti-antigen antibody directly from the clinician and self-administer the immunization or treatment.

### Parts of Plasma

In an exemplary embodiment, Fab/Fab(2) containing anti-antigen antibody fragments or total Ig anti-antigen antibody is used for immunization or treatment. Total immunoglobulin molecule can be fragmented using proteolytic treatment. Proteolytic treatment involves the use of proteolytic enzymes which function in the breakdown of proteins. The proteolytic treatment of the immunoglobulin molecule can result in two Fab fragments and one Fc fragment (proteolytic treatment with papain) as well as two linked Fab fragments [Fab(2)] and one Fc fragment (proteolytic treatment with pepsin). Administering only Fab or Fab(2) is thought to provide comparable, effective, results as compared to administering intact total immunoglobulin molecules. Further, as is described below, the Fab/Fab(2) containing anti-antigen antibody fragments or total Ig anti-antigen antibody may be recombinant. Thus, according to the teachings of the present invention, recombinant Fab/Fab(2) containing anti-antigen antibody fragments or total Ig anti-antigen antibody is prepared.

### Dosage, Timing and Potentiation

The methods of the present invention may involve administering the recombinant Fab/Fab(2) containing anti-antigen antibody fragments or total Ig anti-antigen antibodies to a patient. Immunoglobulin immunization or treatments may be administered using transfusion therapy. One type of transfusion therapy, intravenous immune globulin (IVIG) therapy, involves administering solutions intravenously. To be administered intravenously, the solutions predominantly comprise small molecular weight complexes. Accordingly, the immunization or treatment of the present invention may be administered following the same methodologies as IVIG therapy. Thus, in an exemplary embodiment, Fab/Fab(2) containing anti-antigen antibody fragments or total Ig anti-antigen antibody is administered intravenously.

Another type of transfusion therapy, intramuscular immune globulin (IG) therapy, involves administering solutions intramuscularly. The solutions may comprise high molecular complexes. While solutions comprising high molecular weight complexes may be suitable for IG therapy, the use of such solutions in IVIG therapy would be dangerous. Accordingly, the immunization or treatment of the present invention may be administered following the same methodologies as IG therapy. Thus, in an exemplary embodiment, Fab/Fab(2) containing anti-antigen antibody fragments or total Ig anti-antigen antibody is administered intramuscularly.

While immunization or treatment dosage may be standardized according to easily ascertainable patient characteristics, such as body weight or age, other patient characteristics may be factored into determining proper dosing. The other characteristics include, but are not limited to, the severity of the condition being addressed, the vital statistics of the patient, and the like. Typically, immunization or treatment begins by administering doses less than the determined optimum dosage. The dosages may be increased incrementally until the desired effect is achieved. In an exemplary embodiment wherein immunization or treatment is administered intravenously or intramuscularly, dosage is determined based on the body weight of the patient. The immunization or treatment may be administered to the patient in a dosage amount of between about one picogram (pg) per kilogram (kg) per day (d) (pg/kg/d) to about four grams (g) per kg per d (g/kg/d), wherein the kg value represents the weight of the patient. Further, the solution may be administered to the patient in a dosage amount of between about 75 micrograms (μg) per kg per d (μg/kg/d) to about 4000 milligrams (mg) per kg per d (mg/kg/d). Immunization or treatment may be administered for up to about seven days, although the time may vary depending on factors such as the dosage and the condition of the patient. Immunization or treatment may be repeated every about one to about six months from the initial administration.

For immunization or treatment administered intravenously or intramuscularly, the solutions must be prepared in a suitable, injectable and sterile, form. Suitable injectable forms include, but are not limited to, aqueous solutions and dispersions prepared in carriers such as water, ethanol, glycerol, propylene glycol, liquid polyethylene glycol, vegetable oils, albumin, and the like. Further, the solutions should be prepared and stored in a sterile form and be adequately protected against contamination by microorganisms, such as fungi, bacteria and viruses. Contamination may be prevented by the use of antimicrobial agents such as parabens, chlorobutanol, phenol, sorbic acid, thimerosal, and the like.

In an exemplary embodiment, Fab/Fab(2) containing anti-antigen antibody fragments or total Ig anti-antigen antibody is administered to the patient as an inhalant. The inhalant may be in the form of an aerosol. Fab/Fab(2) containing anti-antigen antibody fragments or total Ig anti-antigen antibody administered as an inhalant allows for the direct treatment of areas of the respiratory tract. Thus, administering Fab/Fab(2) containing anti-antigen antibody fragments or total Ig anti-antigen antibody in the form of an inhalant is useful for, but not limited to, the treatment of respiratory disorders or diseases, for example, antigen mediated respiratory symptoms (e.g., CoViD related respiratory insufficiency).

In the embodiment wherein Fab/Fab(2) containing anti-antigen antibody fragments or total Ig anti-antigen antibody is administered as an inhalant, the Fab/Fab(2) containing anti-antigen antibody fragments or total Ig anti-antigen antibody should be contained in, or formed into, particles of a size sufficiently small to pass through the mouth and larynx upon inhalation and into the bronchi and alveoli of the lungs. The particles should have a size in the range of about one to about ten microns in diameter.

In a further exemplary embodiment, Fab/Fab(2) containing anti-antigen antibody fragments or total Ig anti-antigen antibody is administered to the patient topically. Topical applications are particularly useful for direct localized treatment. Topical applications may include the application of topical treatments, including but not limited to, ointments, creams, transdermal patches, as well as any combination of the foregoing topical treatments. Ointments or creams may be prepared comprising Fab/Fab(2) containing anti-antigen antibody fragments or total Ig anti-antigen antibody and a suitable ointment or cream delivery medium. The ointment or cream may be applied to the areas of the patient requiring the treatment. The Fab/Fab(2) containing anti-antigen antibody fragments or total Ig anti-antigen antibody contained in the ointment or cream will diffuse transdermally into the body of the patient.

Additionally, as mentioned above, Fab/Fab(2) containing anti-antigen antibody fragments or total Ig anti-antigen antibody may be administered using a transdermal patch. The transdermal patch may be worn on the skin of the patient like a bandage. The transdermal patch allows for a prolonged administration. For example, the patient may wear the transdermal patch for a plurality of hours and receive low dose administration throughout that period. Other applicable methods may be used in accordance with the teachings of the present invention. For example, a solution comprising Fab/Fab(2) containing anti-antigen antibody fragments or total Ig anti-antigen antibody may be injected subcutaneously.

The foregoing techniques are provided merely as exemplary methodologies for administration to a patient and it is to be understood that the teachings of the present invention are generally applicable to any suitable methodology and should not be limited to any particular techniques described herein.

Although illustrative embodiments of the present invention have been described herein, it is to be understood that the invention is not limited to those precise embodiments, and that various other changes and modifications may be made by one skilled in the art without departing from the scope or spirit of the invention.

1. A method of inhibiting a viral infection, the method comprising:
a) obtaining a blood product from a first subject wherein the blood product has anti-viral immunoglobulins against a virus,
wherein the blood product is selected from the group consisting of: serum; plasma; and one or more immunoglobulins with or without their corresponding soluble receptors, and fragments thereof; and
b) administering an immunization effective amount of the blood product obtained from the first subject to a second subject who is at risk of becoming infected with the same or similar type of virus,
wherein the viral infection in the second subject is inhibited.

2. The method according to claim 1, wherein the first subject has recovered from an infection by the virus, or has been immunized against the virus.

3. The method of claim 1 wherein inhibiting a viral infection comprises preventing a viral infection.

4. The method of claim 1 wherein the blood product is irradiated prior to administration to the second subject.

5. The method of claim 1, wherein the virus is a coronavirus, HIV, H1N1, H5N1, Powassan virus, Zika virus, chikungunya virus, dengue virus, West Nile virus, herpesvirus, norovirus, parvovirus, human papillomavirus, respiratory syncytial virus, influenza virus, SARS-CoV-2, MERS-CoV, avian flu virus, Ebola virus, influenza A virus, SARS virus, hepatitis virus, measles virus, rubella virus, chickenpox virus, or yellow fever virus.

6. The method of claim 1, further comprising removing a corresponding amount of serum and/or plasma from the second subject prior to administering the serum and/or plasma obtained from the first subject.

7. The method of claim 1, further comprising removing a corresponding amount of serum and/or plasma from the second subject substantially simultaneously with administering the serum and/or plasma obtained from the first subject.

8. The method of claim 1, wherein the one or more immunoglobulins comprise at least one of immunoglobulin G (IgG), immunoglobulin M (IgM), immunoglobulin A (IgA), immunoglobulin E (IgE), and immunoglobulin D (IgD) and fragments thereof.

9. The method of claim 8 wherein the immunoglobulins and/or fragments thereof are selected from the group consisting of antibody binding fragment Fab, antibody subclasses IgG1, IgG2, IgG3, IgG4, and combinations thereof.

10. The method of claim 1, wherein an immunization effective amount of one or more immunoglobulins and one or more soluble receptors corresponding to each immunoglobulin comprises an immunization effective amount of anti-virus specific immunoglobulin.

11. The method of claim 5, wherein the virus is SARS-CoV-2/CoViD-19, the blood product is serum/plasma and the immunization effective amount is about 5 ml to about 450 mL.

12. The method of claim 5, wherein the virus is SARS-CoV-2/CoViD-19, the blood product is serum/plasma and the immunization effective amount is about one picogram per kilogram per day to about four grams per kg per day of antigen-specific immunoglobulin.

13. The method of claim 5, wherein the virus is SARS-CoV-2/CoViD-19, the blood product is serum/plasma and the immunization effective amount is administered at a single or multiple interval sequence from single or multiple subjects who have recovered from a type of virus infection.

14. The method of claim 1, wherein a blood product obtained from the first subject is administered to a plurality of subjects at risk of contracting the same or similar type of virus.

15. The method of claim 1, further comprising administering conventional vaccination protocols including immunological stimulators and/or boosters of specific immune pathways.

16. The method of claim 1, further comprising administering a chimeric or engineered form of the antibody

17. The method of claim 16 wherein the engineered form of the antibody is an Fc component fused with a SARS-Cov-2/CoViD-19 viral antigen in place of Fab.

18. A method of treating viral infections, the method comprising the steps of:
obtaining an amount of at least one blood product from at least a first patient with a type of virus infection; and
administering the at least one of blood product obtained from the at least first patient to at least a second patient with the same type of virus, wherein administering the at least one blood product obtained from the at least first patient to at least a second patient with the same type of virus comprises administering a therapeutically effective amount of one or more immunoglobulins and/or one or more soluble receptors corresponding to each immunoglobulin, and wherein administering a therapeutically effective amount of one or more immunoglobulins and/or one or more soluble receptors corresponding to each immunoglobulin comprises enabling reconstitution of a humoral immune system of the at least a second patient to treat the virus.

19. The method of claim 18, wherein a therapeutically effective amount of one or more immunoglobulins and/or one or more soluble receptors corresponding to each immunoglobulin comprises a therapeutically effective amount of anti-virus specific immunoglobulin.

20. The method of claim 18, wherein enabling reconstitution of a humoral immune system of the at least a second patient comprises neutralizing and/or destroying free virus, and/or virus infected cells and/or alleviating virus progression.
